(19) United States
(12) Patent Application Publication
(10) Pub. No.: US 2013/0089506 A1
(43) Pub. Date: Apr. 11, 2013

(54) ORAL CARE COMPOSITIONS AND METHODS

(71) Applicants: Wei Wang, East Brunswick, NJ (US); James R. Brown, Edison, NJ (US); Harsh M. Trivedi, Hillsborough, NJ (US); Joe Vazquez, Hamilton, NJ (US)

(72) Inventors: Wei Wang, East Brunswick, NJ (US); James R. Brown, Edison, NJ (US); Harsh M. Trivedi, Hillsborough, NJ (US); Joe Vazquez, Hamilton, NJ (US)

(73) Assignee: COLGATE-PALMOLIVE COMPANY, New York, NY (US)

(21) Appl. No.: 13/701,227

(22) PCT Filed: Jun. 24, 2010
(86) PCT No.: PCT/US10/39725

§ 371 Date: Nov. 30, 2012

(51) Int. Cl. A61K 8/97; A61Q 11/00

(52) U.S. Cl. CPC A61K 8/97; A61Q 11/00
USPC 424/58

(57) ABSTRACT

Disclosed are oral care compositions and the use of such oral care compositions for inhibiting co-aggregation of oral bacteria and inhibiting bacterial growth. Also disclosed are methods for inhibiting co-aggregation of oral bacteria and inhibiting bacterial growth. The oral care composition includes cranberry extract non-dialyzable material, in which the cranberry extract non-dialyzable material is present in an amount effective to inhibit co-aggregation of oral bacteria and/or inhibit bacterial growth.

### BACKGROUND OF THE INVENTION

The present invention relates generally to oral compositions useful for enhancing oral hygiene, and more particularly, to cranberry extract non-dialyzable material (NDM) containing oral compositions having enhanced anti-plaque effectiveness.

Adhesion of bacteria to each other as well as to oral surfaces is one of the major factors leading to dental plaque as well as caries and periodontal diseases. It would therefore be helpful to have anti-aggregation compounds that can interrupt microbial adhesion and aggregation.

Cranberry extract non-dialyzable material (NDM) is a high molecular weight, material derived from cranberry juice as described in Ofek, I., Goldhar J. and Sharon N. Anti-Escherichia coli adhesion activity of cranberry and blueberry juices. Adv. Exp. Med. Biol. 1996; 408: 179-183, and in U.S. Pat. Nos. 6,303,125, and 6,843,993, each patent incorporated by reference herein in their entirety.

U.S. Pat. No. 5,683,678, the disclosure of which is incorporated by reference herein in its entirety, discloses anthocyanins isolated from cranberries. Weiss, E., Lev-Dor, R., Kashanm, Y., Goldhar, J., Sharon, N. and Ofek, I. JADA, 129, 1719 (1998) disclose the inhibition of co-aggregation of a large proportion of dental plaque bacteria by cranberry extract NDM. Weiss et al. also disclose an in vitro assay to test the ability of cranberry NDM to inhibit or reverse co-aggregation.

U.S. Pat. Nos. 5,840,322, 6,303,125 and 6,843,993, the disclosures of which are incorporated by reference herein in their entireties, disclose an oral composition comprising cranberry extract NDM that reverses co-aggregation in an in vitro assay at a concentration of 1250 ng/ml. A cranberry extract NDM mouthwash was found to reduce total bacterial counts. However, no change in plaque and gingival indices were observed in a clinical trial that examined the effect of a cranberry extract NDM mouthwash, and the results did not suggest any clinical advantage over standard mouthwashes. Weiss, E., Kozlovsky, A., Steiberg, D., Lev-Dor, R., Greenstein, R., Feldman, M., Sharon, N. and Ofek, I., FEMS Microbiology Letters 232, (2004), p 89-92.

### SUMMARY OF THE INVENTION

There is a need in the art to provide an oral care composition capable of inhibiting bacterial co-aggregation and reducing the accumulation of plaque.

In a first aspect the present invention provides an oral care composition comprising cranberry extract non-dialyzable material and an orally acceptable vehicle, wherein the cranberry extract non-dialyzable material is present in an amount effective to inhibit bacterial co-aggregation. It is preferred that the oral care composition does not contain an ingredient or component that deactivates the cranberry extract non-dialyzable material. In another aspect, the invention provides a method of inhibiting bacterial co-aggregation in the oral cavity comprising applying to the oral cavity an oral care composition comprising an orally acceptable vehicle containing therein an amount of cranberry extract non-dialyzable material effective to inhibit bacterial co-aggregation.

### DETAILED DESCRIPTION

It should be understood that the detailed description and specific examples, while indicating embodiments of the invention, are intended for purposes of illustration only and are not intended to limit the scope of the invention.

The following definitions and non-limiting guidelines must be considered in reviewing the description of this invention set forth herein. The headings (such as “Introduction” and “Summary,”) and sub-headings (such as “Compositions” and “Methods”) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof. Classification or discussion of a material within a section of this specification as having a particular utility (e.g., as being an “active” or a “carrier” ingredient) is made for convenience, and no inference should be drawn that the material must necessarily or solely function in accordance with its classification herein when it is used in any given composition.

The citation of references herein does not constitute an admission that those references are prior art or have any relevance to the patentability of the invention disclosed herein. Any discussion of the content of references cited in the Introduction is intended merely to provide a general summary of assertions made by the authors of the references, and does not constitute an admission as to the accuracy of the content of such references.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations the stated of features. Specific Examples are provided for illustrative purposes of how to make and use the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention. In addition, the compositions and the methods may comprise, consist essentially of, or consist of the elements described therein.

As used herein, the word “include,” and its variants, is intended to be non-limiting, such that recitation of items in a list is not to the exclusion of other like items that may also be useful in the materials, compositions, devices, and methods of this invention.

Throughout this description and claims, the disclosure of a certain numerical value (e.g., temperature, weight percent of components, etc.) is meant to denote that value, plus or minus an additional value that would be understood by persons having ordinary skill in the art, depending on the variable and the degree of measurement error typically associated with that value. For example, a given temperature would be understood by a person having ordinary skill in the art to include up to 10% variability, given the instrument used to measure the temperature.

The expression “co-aggregation” refers to the aggregation/adhesion of two or more bacteria, including bacteria of different species, and inhibition of co-aggregation or adhesion generally refers to prevention of the initial adhesion or aggregation of the bacteria.

The inventive compositions inhibit the co-aggregation of one or more bacteria selected from Streptococcus oralis, Fusobacterium nucleatum, Actinomyces maeslundii, A. viscosus and S. mutan. Other bacteria that may co-aggregate in the oral cavity are also within the scope of the invention.

In the method of inhibiting bacterial co-aggregation the inventive compositions may constitute an integral part of the mouthrinse, toothpaste, dental cream or gel, or tooth powder and applied during regular brushing, or the compositions may be formulated and packaged as a separate treatment and applied separately before, after, and/or in between regular brushing times. The applied compositions may be applied by brushing, rinsing, chewing, and with other means known in the art.

Compositions

In an embodiment, the present invention provides an oral care composition comprising cranberry extract non-dialyzable material and an orally acceptable vehicle, wherein the cranberry extract non-dialyzable material is present in an amount effective to inhibit bacterial co-aggregation. In another aspect, the cranberry extract NDM is present in the composition in an amount effective to inhibit and/or prevent a bacterial co-aggregation in the oral cavity. Preferably, the cranberry extract NDM is present in an amount suitable to prevent or treat a condition caused by bacterial co-aggregation, such as a condition selected from dental plaque, tooth decay, halitosis, periodontal disease and gingivitis. Advantageously, the cranberry extract non-dialyzable material is present in the composition at a concentration of 0.08-1.33 mg/ml. In a preferred embodiment, the cranberry extract non-dialyzable material is preferably present at a concentration of about 0.3% by weight.

The composition according to the present invention inhibits bacterial co-aggregation in the oral cavity. The components of standard oral composition formulations can interfere with the efficacy of cranberry extract NDM at inhibiting bacterial co-aggregation. Some of these components deactivate the cranberry extract NDM, and as a consequence, while some documents have disclosed the use of cranberry extract NDM as having anti-bacterial efficacy, when tested, the compositions did not in fact provide any improved efficacy when compared to standard mouthwash formulations that did not contain the cranberry extract NDM. The inventors discovered that some of the ingredients deactivated the cranberry extract NDM. Accordingly, preferred embodiments of the present invention provide compositions that do not include components that deactivate the cranberry extract NDM. The present inventors have surprisingly found a composition comprising cranberry extract NDM that effectively inhibits bacterial co-aggregation and does not interfere with the activity of cranberry extract NDM.

The present inventors have found that a composition comprising cranberry extract non-dialyzable material has an inhibitory effect on the co-aggregation of oral bacteria. It was found that certain components of standard oral compositions reduce the ability of cranberry extract non-dialyzable material to inhibit bacterial co-aggregation, making it ineffective at reducing bacterial growth. While not intending on being bound by any theory of operation, the present inventors discovered that oral compositions comprising surfactants inhibit the ability of cranberry extract non-dialyzable material to inhibit bacterial co-aggregation. Specifically, surfactants such as poloxamers inhibit the activity of cranberry extract NDM. Other components such as certain flavorants and certain essential oils present in conventional amounts. These components can be used in the context of the present invention, but in amounts lower than that typically employed in mouthwash formulations. A person having ordinary skill in the art can readily determine which ingredients commonly used in mouthwash foimulations (and their respective concentrations) inhibit or interfere with the activity of cranberry extract NDM, using the guidelines provided herein.

In an embodiment, the orally acceptable vehicle is a combination of water, alcohol and one or more humectants. In a preferred embodiment, the alcohol is ethanol. The oral compositions also preferably include one or more humectants selected from sorbitol and glycerin and combinations thereof.

The composition according to the present invention also may comprise one or more further agents typically selected from an anti-plaque agent, a whitening agent, antibacterial agent, cleaning agent, a flavouring agent, a sweetening agent, adhesion agents, surfactants, foam modulators, abrasives, pH modifying agents, humectants, mouth feel agents, colorants, abrasive, tartar control (anticalculus) agent, fluoride ion source, saliva stimulating agent, nutrient and combinations thereof. Various components that may be added to the composition include, for example, a sweetening agent such as saccharin, or sodium saccharin, alcohols such as ethanol, fluoride ion sources such as sodium fluoride, as well as glycerine, sorbitol, propylene glycol, polyethylene glycols, alkyl polyglycoside (APG), polysorbate, PEG40, castor oil, menthol, and the like.

Flavorants among those useful herein include any material or mixture of materials operable to enhance the taste of the composition. Any orally acceptable natural or synthetic flavorant can be used, such as flavoring oils, flavoring aldehydes, esters, alcohols, similar materials, and combinations thereof. Flavorants include vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants, and mixtures thereof. Also encompassed within flavorants herein are ingredients that provide fragrance and/or other sensory effect in the mouth, including cooling or warming effects. Such ingredients include menthol, menthyl acetate, menthyl lactate, camphor, eucalyptus oil, eucalyptol, anethole, eugenol, cassia, oxanone, [alpha]-irisone, propenyl guaiethol, thymol, linalool, benzaldehyde, cinnamaldehyde, N-ethyl-p-menthan-3-carboxamine, N,2,3-trimethyl-2-isopropylbutanamide, 3-1-menthoxypropane-1,2-diol, cinnamaldehyde glycerol acetal (CGA), methone glycerol acetal (MGA), and mixtures thereof. One or more flavorants are optionally present in a total amount of about 0.01% to about 5%, optionally in various embodiments from about 0.05 to about 2%, from about 0.1% to about 2.5%, and from about 0.1 to about 0.5%.

Sweetening agents among those useful herein include dextrose, polydextrose, sucrose, maltose, dextrin, dried invert sugar, mannose, xylose, ribose, fructose, levulose, galactose, corn syrup, partially hydrolyzed starch, hydrogenated starch hydrolysate, sorbitol, mannitol, xylitol, maltitol, isomalt, aspartame, neotame, saccharin and salts thereof, sucralose, dipeptide-based intense sweeteners, cyclamates, dihydrochalcones, and mixtures thereof.

Mouth-feel agents include materials imparting a desirable texture or other feeling during use of the composition. These may include agglomerated silica particles that are designed to break down with agitation, such as SORBOSIL® BFG series, (e.g., BFG 10, BFG 50, BFG 100, etc.), CBT60S, CBT70, or AC33/43 silicas, commercially available from PQ Corporation, Valley Forge, Pa.

Colorants among those useful herein include pigments, dyes, lakes and agents imparting a particular luster or reflectivity such as pearling agents. In various embodiments, colorants are operable to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including FD&C dyes and pigments, talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride, and mixtures thereof. One or more colorants are optionally present in a total amount of about 0.001% to about 20%, for example about 0.01% to about 10% or about 0.1% to about 5%.

The compositions of the present invention may further comprise an optional abrasive useful, for example, as a polishing agent. Any orally acceptable abrasive can be used, but type, fineness, particle size and amount of abrasive should be selected so that tooth enamel is not excessively abraded in normal use of the composition. Suitable optional abrasives include silica, for example in the form of precipitated silica or as admixed with alumina, insoluble phosphates, calcium carbonate, and mixtures thereof. Among insoluble phosphates useful as abrasives are orthophosphates, polymetaphosphates and pyrophosphates. Illustrative examples are dicalcium orthophosphate dihydrate, calcium pyrophosphate, calcium pyrophosphate, tricalcium phosphate, calcium polymetaphosphate and insoluble sodium polymetaphosphate.

The compositions of the present invention optionally comprise a tartar control (anticalculus) agent. Tartar control agents among those useful herein include salts of any of these agents, for example their alkali metal and ammonium salts: phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and. Useful inorganic phosphate and polyphosphate salts include monobasic, dibasic and tribasic sodium phosphates, sodium tripolyphosphate, tetrapolyphosphate, mono-, di-, tri- and tetrasodium pyrophosphates, sodium trimetaphosphate, sodium hexametaphosphate and mixtures thereof.

The compositions of the present invention optionally comprise a fluoride ion source and useful, for example, as an anti-caries agent. Any orally acceptable particulated fluoride ion source can be used, including potassium, sodium and ammonium fluorides and monofluorophosphates, stannous fluoride, indium fluoride, amine fluorides such as olaflur (N′-octadecyltrimethylendiamine-N,N,N-tris(2-ethanol)-dihydrofluoride), and mixtures thereof. One or more fluoride ion sources are optionally present in an amount providing a clinically efficacious amount of soluble fluoride ion to the oral composition.

The compositions of the present invention optionally comprise a saliva stimulating agent useful, for example, in amelioration of dry mouth. Any orally acceptable saliva stimulating agent can be used, including without limitation food acids such as citric, lactic, malic, succinic, ascorbic, adipic, fumaric and tartaric acids, and mixtures thereof. One or more saliva stimulating agents are optionally present in saliva stimulating effective total amount.

The compositions of the present invention optionally comprise a nutrient. Suitable nutrients include vitamins, minerals, amino acids, and mixtures thereof. Vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Nutritional supplements include amino acids (such as L-tryptophane, L-lysine, methionine, threonine, levocarnitine and L-carnitine), lipotropics (such as choline, inositol, betaine, and linoleic acid), and mixtures thereof.

In various embodiments, the oral composition according to the present invention is not intentionally swallowed, but is rather retained in the oral cavity for a time sufficient to effect the intended utility. In other portable embodiments (such as a lozenge, mint, bead, wafer, liquid formulated for oral application from a small portable nebulizer, liquid formulated for oral application from a small portable drop-generating bottle, or a soft pliable tablet), the oral composition is intentionally swallowed, optionally after retention in the oral cavity for a time sufficient to effect intended utility.

The oral care compositions of the various embodiments preferably are in the form of a dentifrice. The term “dentifrice” as used throughout this description, denotes a paste, gel, or liquid formulation. The dentifrice may be in any desired form, such as toothpaste; (including deep striped, surface striped, multi-layered, having a gel surround the paste); powder; beads; mouthwash; mouth rinses; lozenge; dental gel; a periodontal gel; a liquid suitable for painting a dental surface; a chewing gum; a dissolvable, partially dissolvable or non-dissolvable film or strip; a wafer; a wipe or towelette; an implant; a foam; a troche; a dental floss, liquid formulated for oral application in a small portable nebulizer (spray bottle), liquid formulated for oral application in a small portable drop-generating bottle, a soft pliable tablet (“chewie”), or any combinations thereof. As used herein, an “orally acceptable carrier” refers to a material or combination of materials that are safe for use in the compositions of the present invention, commensurate with a reasonable benefit/risk ratio.

The expression “orally acceptable vehicle” or “orally acceptable carrier” used in the context of the present invention means any vehicle useful in formulating any of the dentifrices described above. Suitable orally acceptable vehicles include, for example, one or more of the following: a solvent, an alkaline agent, a humectant, a thickener, a surfactant, an abrasive, an anti-calculus agent, a colorant, a flavoring agent, a dye, a potassium containing salt, an anti-bacterial agent, desensitizing agents, stain reducing agents, and mixtures thereof.

The present invention also provides portable dose article comprising an oral care composition as defined above, wherein the portable dose article is selected from a lozenge, a mint, a bead, a wafer, a small portable nebulizer containing said admixture in liquid formulated for oral application as a spray, a small portable bottle containing said admixture in liquid formulated for oral application as a drop, and a soft pliable tablet.

Preferably, specific materials and compositions to be used in this invention are, accordingly, pharmaceutically- or cosmetically-acceptable, clinically effective, and/or clinically efficacious. As used herein, such a “pharmaceutically acceptable” or “cosmetically acceptable”, “clinically effective”, and/or “clinically efficacious” component is one that is suitable for use with humans and/or animals and is provided in an appropriate amount (a clinically efficacious amount) to provide the desired therapeutic, prophylactic, sensory, decorative, or cosmetic benefit without undue adverse side effects (such as toxicity, irritation, and allergic response) commensurate with a reasonable benefit/risk ratio.

The cranberry extract non-dialyzable material (NDM) is derived from cranberry juice concentrate. Cranberry juice contains high molecular weight materials (NDM) that inhibit bacterial adhesion to host cells as well as the co-aggregation of many oral bacteria. The cranberry extract NDM was prepared according to a method described by Weiss E; Lev-Dor, R.; Kashmamn, Y.; Goldhar, J.; Sharon, N.; Ofek, Itzhak, J. Am. Dent. Assoc. 129, 1719 (1998).

Methods of Use

The composition according to the present invention may be administered to or applied to a human or other animal subject. The composition may be suitable for administration or application to the oral cavity of a human or animal subject for inhibiting bacterial co-aggregation. Accordingly, the present invention provides a composition as defined above for use as a medicament or cosmetic agent.

The present invention also provides an oral care composition comprising cranberry extract non-dialyzable material and an orally acceptable vehicle, wherein the cranberry extract non-dialyzable material is present in an amount effective to inhibit bacterial co-aggregation. The present invention also provides a method of inhibiting bacterial co-aggregation in the oral cavity comprising applying to the oral cavity an oral care composition comprising an orally acceptable vehicle containing therein an amount of cranberry extract non-dialyzable material effective to inhibit bacterial co-aggregation.

A composition comprising cranberry extract NDM and an orally acceptable vehicle is also capable of significantly inhibiting bacterial co-aggregation. The composition is particularly useful for inhibiting bacterial co-aggregation in the oral cavity. A medicament comprising the composition according to the present invention may be administered to a patient.

Each and every reference cited herein is hereby incorporated by reference in its entirety. Various embodiments now will be described with reference to the following non-limiting examples

### SPECIFIC EMBODIMENTS OF THE INVENTION

### Example 1

### Mouthwash Formulation Containing Cranberry Extract NDM

The cranberry extract NDM was prepared according to a method described by Weiss, et al. Am. Dent. Assoc. 129(12), 1719 (1998). The cranberry extract NDM was obtained by dialyzing cranberry juice through a high molecular weight cut-off dialysis bag. The substance left in the bag that does not dialyze out is the non-dialyzable material (NDM). The cranberry extract NDM was formulated in a mouthwash composition (shown in Table 1).

TABLE 1
Mouthwash formula containing cranberry
extract NDM as active ingredient

| Component | Weight % |
|---|---|
| 96% Ethanol (95% same) | 6.00 |
| Cranberry extract NDM | 0.30 |
| CP purified water | 73.58 |
| Sodium saccharin | 0.02 |
| Sodium fluoride - USP | 0.05 |
| Sodium benzoate | 0.05 |
| Sorbitol 70% solution (NB, NC) | 10.00 |
| Glycerin 99% Organic Kosher | 10.00 |
| TOTAL | 100.00 |

An in vitro assay showed that the cranberry extract NDM mouthwash has efficacy against co-aggregation of bacterial pair S. sangius and F. nucleatuin when diluted 8-fold.

An in vitro assay also showed that this mouthwash had an inhibitory effect on growth of A. viscosus when diluted 25 fold (Table 4 and Table 5).

### Example 2

### Mouthwash Formulation That Deactivates Cranberry Extract NDM

An example of a formulation that will deactivate cranberry NDM

TABLE 2
Example of a cranberry extract NDM formulation that
does not inhibit bacterial co-aggregation or growth

| Component | Weight % |
|---|---|
| 95% Ethanol | 6.00 |
| Cranberry extract NDM | 0.35 |
| CP purified water | 71.53 |
| Sodium saccharin | 0.02 |
| Sodium fluoride - USP | 0.05 |
| Sodium benzoate | 0.05 |
| Poloxamer 338 NF | 1.00 |
| Poloxamer 407 NF | 1.00 |
| Sorbitol 70% solution (NB, NC) | 10.00 |
| Glycerin 99% Organic Kosher | 10.00 |
| TOTAL | 100.00 |

An in vitro assay showed that the mouthwash formulation shown in Table 2 did not effectively inhibit bacterial co-aggregation. Accordingly, the presence of poloxamer inhibited or interfered with the activity of the cranberry extract NDM. Persons having ordinary skill in the art can readily test other components and their respective amounts using the afore-mentioned protocol to ascertain other commonly used components in mouthwash formulations that inhibit or otherwise interfere with the activity of the cranberry extract NDM.

The specific experimental method of the anti co-aggregation assay are described in Weiss E I, Lev-Dor R, Kashamn Y, Goldhar J, Sharon N, Ofek I, “Inhibiting interspecies co-aggregation of plaque bacteria with a cranberry juice constituent,” J. Am. Dent. Assoc. 1988 December; 129(12): 1719-23.

### Example 3

### Anti Co-Aggregation Assay

A mouthwash formula containing 0.3% cranberry extract NDM has both an anti co-aggregation effect and a bacterial growth inhibition effect. The anti co-aggregation results shown in Table 3 indicate that cranberry extract NDM effectively inhibits co-aggregation of bacterial pairs.

The specific experimental method of the anti co-aggregation assay are described in Weiss EI, Lev-Dor R, Kashamn Y, Goldhar J, Sharon N, Ofek I, “Inhibiting interspecies co-aggregation of plaque bacteria with a cranberry juice constituent,” J. Am. Dent. Assoc. 1988 December; 129(12): 1719-23.

The results shown in Table 3 indicate that cranberry extract NDM in neat solution is an efficacious inhibitor of bacterial co-aggregation.

TABLE 3
Anti co-aggregation test result of Cranberry
extract NDM in neat solution

| Final concentration | NDM I | NDM I | NDM II | NDM II |
|---|---|---|---|---|
| (mg/ml) | Act/Sm | So/Fn | Act/Sm | So/Fn |
| 1.33 | 0 | 0 | 0 | 0 |
| 0.66 | 0 | 0 | 0 | 0 |
| 0.33 | 0 | 0 | 0 | 0 |
| 0.16 | 1 | 0 | 1 | 0 |
| 0.08 | 3 | 4 | 3 | 4 |
| Negative control | 3 | 4 | 3 | 4 |

Note:
0 = no co-aggregation (complete inhibition); 4 = full co-aggregation (no inhibition)
So/Fn: Streptococcus oralis/Fusobacterium nucleatum
Act/Actinomyces naeslundii/S. mutan

### Example 4

### Growth Inhibition Assay

The bacteria A. viscosus was propagated from a single colony growing on a blood agar plate. It was aseptically transferred to a centrifuge tube containing 30 mL of sterile TSB media. The centrifuge tube was then placed in a 37.5° C. incubator to grow overnight. The following day, the bacterial solution was gram stained for purity and then diluted to an optical density of 0.23 at 610 nanometers on the UV spectrometer. A volume of 9.6 mL of the inoculum was added to Falcon tubes with 0.4 mL of the rinse being tested to result in a final dilution of 1:25 of the rinse. The tubes were then incubated in a shaking water bath at 37.5° C. At specific time intervals, 1 mL was removed from the tubes and placed into a cuvette in order to obtain the UV spectrum.

This in vitro assay showed that the cranberry extract NDM mouthwash formulation had an inhibitory effect on growth of A. viscosus when diluted 25 fold (Table 4 and Table 5).

TABLE 4
Data of growth inhibition test of bacteria A. viscosus.

| Sample | 0 hour | 2 hours | 4 hours | 22 hours | 24 hours |
|---|---|---|---|---|---|
| Water | 0.2341 | 0.3603 | 0.5756 | 1.5848 | 1.7604 |
| Placebo Mouthwash | 0.2341 | 0.3376 | 0.5234 | 1.4558 | 1.5740 |
| Mouthwash with | 0.2341 | 0.1703 | 0.1918 | 0.7879 | 1.0405 |
| 0.3% Cranberry | | | | | |
| Extract NDM | | | | | |

TABLE 5
Percent reduction after 24 hours based on data in Table 4.

| | |
|---|---|
| Placebo Mouthwash | 10.6% |
| Mouthwash w/0.3% | 40.9% |
| Cranberry Extract NDM | |

The invention has been described above with reference to illustrative Examples, but it is to be understood that the invention is not limited to the disclosed embodiments. Alterations and modifications that would occur to one of skill in the art upon reading the specification are also within the scope of the invention, which is defined in the appended claims.

What is claimed is:

1) An oral care composition comprising cranberry extract non-dialyzable material and an orally acceptable vehicle, wherein the cranberry extract non-dialyzable material is present in a concentration of 0.08-1.33 mg/ml, and wherein the composition does not contain a component that deactivates the cranberry extract non-dialyzable material.

2) An oral care according to claim 1, wherein the cranberry extract non-dialyzable material is present in the composition in a concentration of about 0.3% w/w.

3) An oral care composition according to claim 1, wherein the orally acceptable vehicle is selected from one or more of the group consisting of water, alcohol, a humectant, and combinations thereof.

4) An oral care composition according to claim 3, wherein the alcohol is ethanol.

5) An oral care composition according to claim 4, wherein the alcohol is present in a concentration of about 6% w/w.

6) An oral care composition according to claim 3, wherein the humectant is selected from the group consisting of one or more of sorbitol, glycerine, and mixtures thereof.

7) An oral care composition according to claim 6, wherein the humectant is present in a concentration of about 10% w/w.

8) An oral care composition according to claim 1, wherein the composition is a mouthrinse.

9) An oral care composition according to claim 1, wherein the composition further comprises one or more agents selected from the group consisting of an anti-plaque agent, a whitening agent, a preservative, a sweetening agent, a cleaning agent, a flavoring agent, and mixtures thereof.

10) An oral care composition according to claim 1, wherein the composition is in the form of a dentifrice selected from the group consisting of: toothpaste; deep striped toothpaste; surface striped toothpaste; multi-layered toothpaste; a gel surrounding toothpaste; powder; beads; mouthwash; mouth rinses; lozenge; dental gel; a periodontal gel; a liquid suitable for painting a dental surface; a chewing gum; a dissolvable, partially dissolvable or non-dissolvable film or strip; a wafer; a wipe or towelette; an implant; a foam; a troche; a dental floss, liquid formulated for oral application in a small portable bottle; liquid formulated for oral application in a small portable drop-generating bottle; a soft pliable tablet (“chewie”); and combinations thereof.

11) A method of inhibiting bacterial co-aggregation in the oral cavity comprising applying to the oral cavity an oral care composition comprising an orally acceptable vehicle containing therein cranberry extract non-dialyzable material in a concentration of 0.08-1.33 mg/ml, and wherein the composition does not contain a component that deactivates the cranberry extract non-dialyzable material.

12) A method according to claim 11, wherein the cranberry extract non-dialyzable material is present in the composition at a concentration of 0.3% w/w.

13) A method according to claim 11, wherein the orally acceptable vehicle is selected from one or more of the group consisting of water, alcohol, a humectant, and combinations thereof.

14) A method according to claim 13, wherein the alcohol is ethanol.

15) A method according to claim 14, wherein the alcohol is present in a concentration of about 6% w/w.

16) A method according to claim 13, wherein the humectant is selected from the group consisting of one or more of sorbitol, glycerine, and mixtures thereof

17) A method according to claim 16, wherein the humectant is present in a concentration of about 10% w/w.

18) A method according to claim 11, wherein the composition is a mouthrinse.

19) A method according to claim 11, wherein the composition comprises one or more further agents selected from an anti-plaque agent, a whitening agent, a preservative, a sweetening agent, a cleaning agent and a flavoring agent

20) A method according to claim 11, wherein the composition is in the form of a dentifrice selected from the group consisting of: toothpaste; deep striped toothpaste; surface striped toothpaste; multi-layered toothpaste; a gel surrounding toothpaste; powder; beads; mouthwash; mouth rinses; lozenge; dental gel; a periodontal gel; a liquid suitable for painting a dental surface; a chewing gum; a dissolvable, partially dissolvable or non-dissolvable film or strip; a wafer; a wipe or towelette; an implant; a foam; a troche; a dental floss, liquid formulated for oral application in a small portable bottle; liquid formulated for oral application in a small portable drop-generating bottle; a soft pliable tablet (“chewie”); and combinations thereof.

21) An oral care composition comprising an orally acceptable vehicle and an agent for inhibiting bacterial co-aggregation wherein said agent consists essentially of cranberry extract non-dialyzable material in a concentration of 0.08-1.33 mg/ml and wherein the composition does not contain a component that deactivates the cranberry extract non-dialyzable material.
